(19) United States
(12) Patent Application Publication
(10) Pub. No.: US 2003/0215787 A1
(43) Pub. Date: Nov. 20, 2003

(54) Modulators of the CLC-7 chloride channel and methods for their identification and use in the treatment and prevention of osteoporosis and related disease states

(76) Inventors: Wen-Pin Yang, Princeton, NJ (US); Anita C. Knapp-Ryseck, Titusville, NJ (US); Yuli Wu, Newtown, PA (US); Jinwen Huang, West Windsor, NJ (US); Judith Wardwell-Swanson, Princeton Junction, NJ (US); Stephen Glenn Walker, East Windsor, NJ (US); Jian Chen, Princeton, NJ (US); Stephen S. Kalinowski, Levittown, PA (US); Zelin Sheng, Belle Mead, NJ (US); Samuel E. Hellings, Levittown, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/349,670

(22) Filed: Jan. 23, 2003

Related U.S. Application Data
(60) Provisional application No. 60/351,187, filed on Jan. 23, 2002.

(51) Int. Cl. A61K 31/00; C12Q 1/00

(52) U.S. Cl. 435/4; 514/1

(57) ABSTRACT

The present invention relates to compounds which act as modulators of the chloride channel CLC-7, assays for the identification of such compounds and methods of preventing, diagnosing, and treating osteoporosis and related disease states using such compounds.

This application claims the benefit of provisional U.S. Application Serial No. 60/351,187, filed Jan. 23, 2002, which is incorporated herein by reference in its entirety.

### FIELD OF THE INVENTION

The present invention relates to methods for the chloride channel modulators, the identification of such modulators, and their use in the treatment and prevention of disease. Particularly, the present invention relates to methods, including cell based assays, directed to the screening, identification and characterization of compounds useful as modulators of the CLC-7 chloride channel. Assays of the present invention can be used to identify inhibitors or activators of CLC-7 which are useful for aiding in the diagnosis, treatment and prevention of osteoporosis and related disease states.

### BACKGROUND OF RELATED TECHNOLOGY

Osteoporosis is a chronic, degenerative disease characterized by progressive bone loss, and is caused primarily by an imbalance between bone resorption by osteoclast cells and bone formation. Increased bone resorption is the primary cause of type I osteoporosis occurring in post-menopausal women.

The osteoclast is a terminally differentiated cell derived from monocytic/macrophage lineage which resorbs bone as part of the normal process of skeletal modeling and remodeling, and is the principal resorptive cell of bone. In contrast to precursor cells, only fully differentiated mature osteoclasts are able to resorb bone. As activated osteoclasts move over the bone surface to initiate new sites of bone resorption, cytoskeletal rearrangements lead to the formation of unique cell adhesion structures called podosomes, which attach to the bone matrix via intermediate steps. As such, various aspects of osteoclast function have been studied in attempts to identify anti-resorptive compounds. However, known anti-resorptive agents are replete with side-effects.

Numerous ion currents have been identified in osteoclasts using molecular and electrophysiological approaches. These currents and their corresponding channels are known to play important roles in various cell types, and, accordingly, are believed to also play important roles in the membrane potential and volume regulation of osteoclasts. As such, bone resorption by osteoclasts is likely to be effected by these channels. However, the identity and composition of these channels, such as chloride channels, is not fully understood.

Accordingly, there is a continuing need to identify molecular targets, such as genes, whose expression is associated with bone resorption. The identification of such genes permits the development of clones expressing such genes, thereby permitting the identification of compounds capable of modulating the activity of such genes and/or their expression products. Such compounds are expected to exhibit therapeutic utility in the diagnosis and/or treatment of osteoporosis and related disease states. The present invention is directed to meeting these and other needs.

### BRIEF DESCRIPTION OF THE DRAWINGS

FIG. 1 shows a cluster analysis of expression data of osteoclast differentiation using Self-Organizing Map software.

FIG. 2 shows inhibition of bone resorption by specific CLC-7 RNAi.

FIG. 3A shows CLC-7 localization in RAW 264.7 cells by CLC-7-gfp transient transfection of RAW 264.7 cells 48 hours post transfection (1 day post RANK-L stimulation).

FIG. 3B shows CLC-7 localization in RAW 264.7 cells by CLC-7-gfp transient transfection of RAW 264.7 cells 72 hours post transfection (2 days post RANK-L stimulation).

FIG. 4 shows CLC-7 localization in RPE cells by CLC-7-gfp transient transfection of RPE cells 24 hours post transfection.

FIG. 5A shows CLC-7 localization in HEK293 cells by CLC-7-gfp transient transfection of HEK 293 cells 24 hours post transfection.

FIG. 5B shows a 5× digital zoom of the boxed region in FIG. 5A.

FIG. 6 shows the expression CLCs in Raw264 cells by PCR.

FIG. 7 shows an analysis of CLC-7 Stable Cell Lines by CLC-7 expression in transfected HEK293 cells relative to GAPDH.

FIG. 8 shows a Western blot analysis of CLC-7 stable cell lines.

FIG. 9A shows HEK-293 cells transfected with CLC-7 and stained for LAMP-1.

FIG. 9B shows HEK-293 cells transfected with CLC-7 and stained for CLC-7.

FIG. 9C shows an overlay of FIGS. 9A and 9B to show where LAMP-1 and CLC-7 co-localize.

### SUMMARY OF THE INVENTION

Chloride channels play important roles in the functions of plasma membrane and in intracellular organelles, such as mediating charge compensation during electrogenic H+ transport into the resorption microenvironment. Through the use of transcriptional profiling, CLC-7 is identified in the present invention as a chloride channel involved in this process during osteoclast resorption. Inhibiting the activation of CLC-7 reduces the acidification of the resorption pit and, consequently, bone resorption. Accordingly, modulators of CLC-7 are useful in affecting osteoclast activation and bone resorption. Such modulators may be identified using assays of the present invention, and are therefore expected to be useful as therapeutic compounds to treat osteoporosis and related disease states.

CLC-7 target validation studies on modulators identified using methods of the present invention may be carried out using conventional osteoporosis mouse and rat models. Further, such compounds are suitable for use in compositions for the treatment of osteoporosis and related disease states, and may be administered in any conventional manner. The present invention further includes the use of antisense or RNAi therapy.

In one aspect, the present invention relates to an assay for identifying a compound that modulates the activity of the chloride channel CLC-7, including the steps of: (a) providing a cell expressing CLC-7; (b) contacting the cell expressing CLC-7 with a test compound; and (c) determining whether the test compound modulates the activity of CLC-7. The assay can be a cell-based assay or a cell-free assay, such as a detached patch clamping. Further, the test compound may modulate the activity of CLC-7, and may be an activator or inhibitor of CLC-7. The test compound may also bind to CLC-7, and may be useful for the treatment of osteoporosis.

In another aspect, the present invention relates to a method for the treatment of osteoporosis, including administering to a patient in need thereof a therapeutically effective amount of a compound identified by the assay stated above.

In another aspect, the present invention relates to a method for the treatment of osteoporosis, including the steps of: (a) identifying a patient having osteoporosis; and (b) administering to the patient a therapeutically effective amount of a modulator of CLC-7.

In another aspect, the present invention relates to a method for the prevention of osteoporosis, including the steps of: (a) identifying a patient at risk for osteoporosis; and (b) administering to the patient a therapeutically effective amount of a modulator of CLC-7.

In another aspect, the present invention relates to a method of decreasing the differentiation of osteoclast precursor cells into osteoclast cells or decreasing the activity of mature osteoclasts, including contacting the osteoclast precursor cells or mature osteoclasts with a CLC-7 modulator.

In another aspect, the present invention relates to a compound capable of modulating the activity of CLC-7. The compound may be identified by: (a) providing a cell expressing CLC-7; (b) contacting the cell expressing CLC-7 with the compound; and (c) determining whether the compound modulates the activity of CLC-7. The compound may be a CLC-7 inhibitor or activator, and may bind to CLC-7.

### DETAILED DESCRIPTION OF THE INVENTION

The osteoclast is a terminally differentiated cell derived from monocytic/macrophage lineage that resorbs bone as part of the normal process of skeletal remodeling. Increased osteoclastic bone resorption leads to many skeletal disorders, most notably post-menopausal osteoporosis in women and frailty in aging men.

Through development of podosomes, activated osteoclasts move over the bone surface to initiate new sites of bone resorption. These events are initiated preferentially through the interaction of receptor activator of NF-κB ligand (RANKL) with the RANKL receptor present on the osteoclast membrane. RAW264.7 cells may be differentiated into functional osteoclasts upon activation with RANKL. The present invention is directed to the finding that these cells, differentiated to osteoclasts in an in vitro cell model, have upregulated CLC-7 that can be inhibited by chloride channel inhibitors. This inhibition leads to the reduction of acidification and, thus, the reduction of bone resorption.

The reported DNA sequence (SEQ ID NO:1) and amino acid sequence (SEQ ID NO:2) of human CLC-7 is set forth in Tables 1 and 2, respectively. The reported DNA sequence (SEQ ID NO:3) and amino acid sequence (SEQ ID NO:4) of rat CLC-7 is set forth in Tables 3 and 4, respectively. The reported DNA sequence (SEQ ID NO:5) and amino acid sequence (SEQ ID NO:6) of mouse CLC-7 is set forth in Tables 5 and 6, respectively. Human, rat and mouse CLC-7 sequences were obtained from the GenBank database.

One of skill in the art will recognize that CLC-7 suitable for use in the present invention is desirably human, rat or mouse, but may include CLC-7 from any suitable organism. The protein and genomic sequences of these organisms are readily accessed via GenBank or The National Center for Biotechnology Information.

TABLE 1

Nucleotide Sequence of Human CLC-7:
GenBank Accession No. AF224741

(SEQ ID NO:1)

GCCGGCGCTTCCCGGCCGGTGTCGCTCCGCGGCGGGCCATGGCCAACGTC
TCTAAGAAGGTGTCCTGGTCCGGCCGGGACCGGGACGACGAGGAGGCGGC
GCCGCTGCTGCGGAGGACGGCGCGGCCCGGCGGGGGGACGCCGCTGCTGA
ACGGGGCTGGGCCCGGGGCTGCGCGCCAGTCACCACGTTCTGCGCTTTTC
CGAGTCGGACATATGAGCAGCGTGGAGCTGGATGATGAACTTTTGGACCC
GGATATGGACCCTCCACATCCCTTCCCCAAGGAGATCCCACACAACGAGA
AGCTCCTGTCCCTCAAGTATGAGAGCTTGGACTATGACAACAGTGAGAAC
CAGCTGTTCCTGGAGGAGGAGCGGCGGATCAATCACACGGCCTTCCGGAC
GGTGGAGATCAAGCGCTGGGTCATCTGCGCCCTCATTGGGATCCTCACGG
GCCTCGTGGCCTGCTTCATTGACATCGTGGTGGAAAACCTGGCTGGCCTC
AAGTACAGGGTCATCAAGGGCAATATCGACAAGTTCACAGAGAAGGGCGG
ACTGTCCTTCTCCCTGTTGCTGTGGGCCACGCTGAACGCCGCCTTCGTGC
TCGTGGGCTCTGTGATTGTGGCTTTCATAGAGCCGGTGGCTGCTGGCAGC
GGAATCCCCCAGATCAAGTGCTTCCTCAACGGGGTGAAGATCCCCCACGT
GGTGCGGCTCAAGACGTTGGTGATCAAAGTGTCCGGTGTGATCCTGTCCG
TGGTCGGGGGCCTGGCCGTGGGAAAGGAAGGGCCGATGATCCACTCAGGT
TCAGTGATTGCCGCCGGGATCTCTCAGGGAAGGTCAACGTCACTGAAACG
AGATTTCAAGATCTTCGAGTACTTCCGCAGAGACACAGAGAAGCGGGACT
TCGTCTCCGCAGGGGCTGCGGCCGGAGTGTCAGCGGCGTTTGGAGCCCCC
GTGGGTGGGGTCCTGTTCAGCTTGGAGGAGGGTGCGTCCTTCTGGAACCA
GTTCCTGACCTGGAGGATCTTCTTTGCTTCCATGATCTCCACGTTCACCC
TGAATTTTGTTCTGAGCATTTACCACGGGAACATGTGGGACCTGTCCAGC
CCAGGCCTCATCAACTTCGGAAGGTTTGACTCGGAGAAAATGGCCTACAC
GATCCACGAGATCCCGGTCTTCATCGCCATGGGCGTGGTGGGCGGTGTGC
TTGGAGCTGTGTTCAATGCCTTGAACTACTGGCTGACCATGTTTCGAATC
AGGTACATCCACCGGCCCTGCCTGCAGGTGATTGAGGCCGTGCTGGTGGC
CGCCGTCACGGCCACAGTTGCCTTCGTGCTGATCTACTCGTCGCGGGATT
GCCAGCCCCTGCAGGGGGGCTCCATGTCCTACCCGCTGCAGCTCTTTTGT
GCAGATGGCGAGTACAACTCCATGGCTGCGGCCTTCTTCAACACCCCGGA
GAAGAGCGTGGTGAGCCTCTTCCACGACCCGCCAGGCTCCTACAACCCCC
TGACCCTCGGCCTGTTCACGCTGGTCTACTTCTTCCTGGCCTGCTGGACC
TACGGGCTCACGGTGTCTGCCGGGGTCTTCATCCCGTCCCTGCTCATCGG
GGCTGCCTGGGGCCGGCTCTTTGGGATCTCCCTGTCCTACCTCACGGGGG
CGGCGATCTGGGCGGACCCCGGCAAATACGCCCTGATGGGAGCTGCTGCC
CAGCTGGGCGGGATTGTGCGGATGACACTGAGCCTGACCGTCATCATGAT
GGAGGCCACCAGCAACGTGACCTACGGCTTCCCCATCATGCTGGTGCTCA
TGACCGCCAAGATCGTGGGCGACGTCTTCATTGAGGGCCTGTACGACATG
CACATTCAGCTGCAGAGTGTGCCCTTCCTGCACTGGGAGGCCCCGGTCAC
CTCACACTCACTCACTGCCAGGGAGGTGATGAGCACACCAGTGACCTGCC
TGAGGCGGCGTGAGAAGGTCGGCGTCATTGTGGACGTGCTGAGCGACACG
GCGTCCAATCACAACGGCTTCCCCGTGGTGGAGCATGCCGATGACACCCA
GCCTGCCCGGCTCCAGGGCCTGATCCTGCGCTCCCAGCTCATCGTTCTCC
TAAAGCACAAGGTGTTTGTGGAGCGGTCCAACCTGGGCCTGGTACAGCGG
CGCCTGAGGCTGAAGGACTTCCGAGACGCCTACCCGCGCTTCCCACCCAT
CCAGTCCATCCACGTGTCCCAGGACGAGCGGGAGTGCACCATGGACCTCT
CCGAGTTCATGAACCCCTCCCCCTACACGGTGCCCCAGGAGGCGTCGCTC
CCACGGGTGTTCAAGCTGTTCCGGGCCCTGGGCCTGCGGCACCTGGTGGT
GGTGGACAACCGCAATCAGGTTGTCGGGTTGGTGACCAGGAAGGACCTCG
CCAGGTACCGCCTGGGAAAGAGAGGCTTGGAGGAGCTCTCGCTGGCCCAG
ACGTGAGGCCCAGCCCTGCCCATAATGGGCACTGGCGCTGGCACCCCGGC
CCTTCTGCATTTCCTCCCGGAGTCACTGGTTTCTCGGCCCAAACCATGCT
CCCCAGCAGTGGCAATGGCGAGCACCCTGCAGCTGGGCGGGCAGGCGGCA
GGCGCGGAACTGACCCTCTCGCGGGACTGACCCTGTTGTGGGCAGTGGTC
TCCCCCCTTGGCGCCTCCTTGCGCAGGCCCAGCCTCCACTCTCCTCGTCT
AGGTTTCTTTACCTCCAGGGATCAGCTGTGTGTGTGTGACCTCCCTACCG
GGCTATCGGCCTCTTGGGAGCCAGCGGCAGGGCCGGCACCTGCGTGCCTG
TGCCCGTGTGCGTGAGACAGAGCCCTTGCCCCTGCTGCTGCCCCGAGGGC
TGCCCTGCCCTGGAAGGGCCCCTCTGCCTCCACACCAGTGGAGTCTTCGA
GACTTGGGAGCTGCTTGGCCTCATTTTCAGCCATGAGCAGACGGCCTGTG
GTCCCTGGGCCTGAGGCACGGACTCGTAGCACCAGGGTTTGGAGGCTGCG
ACCGCCCCGGAGAGCAGCTTCACACTGGCGCCACAGAGGAGCCCCACGTG
CACTCCCCGGCCTGCATCCGGCTTGGGTACACAGGCCCAGAGGACTGGGG
TGACTCACGGGCCCTGTGCTGTGATGTTGAGAGCTGAGAAAAACCTCCAA
GGCCCTGAGCCCCATGCCCAGCCCTGCCTTGGTCCCCCAATCCCCAGAGC
TTGGAGTCTGGGCCCCACACCCAGCCCTGCCTTGGTCCCTGAGCCTCAAA
GCGTGGAATTGCTGCCCTGTGGACACT

TABLE 2

Amino Acid Sequence of Human CLC-7:
GenBank Accession No. AAF34711

(SEQ ID NO:2)

MANVSKKVSWSGRDRDDEEAAPLLRRTARPGGGTPLLNGAGPGAARQSPR
SALFRVGHMSSVELDDELLDPDMDPPHPFPKEIPHNEKLLSLKYESLDYD
NSENQLFLEEERRINHTAFRTVEIKRWVICALIGILTGLVACFIDIVVEN
LAGLKYRVIKGNIDKFTBKGGLSFSLLLWATLNAAFVLVGSVIVAFWPVA
AGSGIPQIKCFLNGVKIPHVVRLKTLVIKVSGVWSVVGGLAVGKEGPMIH
SGSVIAAGISQGRSTSLKRDFKIFEYFRRDTEKRDFVSAGAAAGVSAAFG
APVGGVLFSLEEGASFWNQFLTWRTFFASMISTFTLNFVLSIYHGNMWDL
SSPGUNFGRFDSEKMAYTIHEIPVFIAMGVVGGVLGAVFNALNYWLTMFR
IRYIHRPCLQVIIEAVLVAAVTATVAFVLIYSSRDCQPLQGGSMSYPLQL
FCADGEYNSMAAAFFNTPEKSVVSLFHDPPGSYNPLTLGLFTLVYFFLAC
WTYGLTVSAGVFIPSLLIGAAWGRLFGISLSYLTGAAIWADPGKYALMGA
AAQLGGIVRMTLSLTVIIMMEATSNVTYGFPIMLVLMTAKIVGDVFIEGL
YDMHIQLQSVPFLHWEAPVTSHSLTAREVMSTPVTCLRRREKVGVIVDVL
SDTASNHNGFPVVEHADDTQPARLQGLILRSQLIVLLKHKVFVERSNLGL
VQRRLRLKDFRDAYPRFPPIQSIIHVSQDERECTMDLSEFMNPSPYTVPQ
EASLPRVFKLFRALGLRHLVVVDNRNQVVGLVTRKDLARYRLGKRGLEEL
SLAQT

TABLE 3

Nucleotide Sequence of Rat CLC-7:
GenBank Accession No. NM031568

(SEQ ID NO:3)

GGGGCGCGGGTCACGGGAACGCTGCCGGGCTGCCGGCTGTTCTTGTGGAG
TTTGGTCCTCAGTGGGCCATGGCCAACGTTTCTAAGAAAGTGTCTTGGTC
CGGCCGAGATCGCGATGACGAGGAGGGGGCGCCGCTGCTTCGAAGGACGG
GGCAACCTGACGAGGAGACGCCGCTGCTGAACGGAGCCGGGCCGGGCGCG
CGCCAGTCTCATTCTGCACTTTTCCGAATTGGACAGATGAACAACGTGGA
GCTGGATGATGAACTCCTGGACCCGGAAGTGGACCCTCCTCACACCTTCC
CCAAGGAGATTCCACACAACGAGAAGCTCCTCTCCCTCAAGTATGAGAGC
CTGGACTATGACAATAGTGAGAATCAGCTCTTCCTGGAGGAGGAAAGACG
AATCAACCACACGGCTTTCCGGACAGTGGAGATCAAGCGCTGGGTTATCT
GTGCCCTCATTGGAATCCTCACAGGCCTAGTAGCCTGCTTCATTGACATT
GTAGTGGAGAACCTGGCAGGCCTCAAGTACCGAGTCATCAAGGACAACAT
CGACAAGTTCACAGAGAAGGGCGGCCTGTCCTTCTCCCTCCTGCTGTGGG
CCACACTGAACTCTGCCTTCGTGCTCGTGGGGTCTGTGATTGTGGCCTTC
ATAGAGCCAGTTGCTGCTGGCAGCGGAATCCCTCAGATCAAGTGCTTCCT
CAATGGGGTGAAGATCCCCCACGTGGTGCGGCTCAAGACGCTGGTGATCA
AGGTGTCTGGCGTGATTCTGTCTGTGGTAGGGGGACTGGCTGTGGGAAAG
GAAGGGCCAATGATCCACTCAGGATCCGTGATTGCTGCAGGGATTTCACA
GGGAAGGTCGACGTCACTCAAGCGAGATTTTAAGATCTTTGAATATTTCC
GCAGAGATACAGAGAAGCGGGATTTTGTCTCAGCTGGAGCTGCAGCTGGA
GTGTCTGCTGCGTTTGGAGCACCTGTGGGTGGGGTCCTGTTCAGCCTGGA
AGAGGGCGCCTCCTTCTGGAATCAGTTCCTGACATGGAGAATTTTCTTTG
CTTCCATGATTTCGACCTTTACACTGAATTTTGTTCTGAGCATCTACCAT
GGAAACATGTGGGACCTGTCCAGCCCTGGCCTCATAAATTTTGGAAGATT
CGACTCAGAGAAAATGGCCTACACAATCCATGAGATTCCTGTCTTCATCG
CCATGGGTGTGGTGGGTGGCATTCTTGGAGCCGTGTTCAATGCCTTGAAT
TACTGGCTAACTATGTTTCGAATCAGGTACATCCACCGGCCCTGCCTCCA
AGTGATTGAGGCCATGCTGGTGGCAGCTGTCACAGCCACAGTTGCATTTG
TCTTGATTTACTCGTCTCGAGATTGCCAGCCCCTGCAGGGGAGCTCCATG
TCCTACCCACTCCAGCTCTTCTGTGCAGATGGCGAATACAACTCAATGGC
CGCAGCCTTCTTTAACACCCCTGAGAAGAGCGTCGTCAGCCTGTTCCACG
ACCCACCAGGCTCCTATAATCCCATGACTCTCGGCCTGTTCACCCTGGTC
TACTTCTTCCTGGCCTGCTGGACCTATGGCCTCACAGTATCTGCTGGTGT
CTTCATCCCATCCCTGCTCATTGGGGCTGCCTGGGGCCGACTCTTTGGCA
TCTCCATGTCCTACCTCACAGGAGCAGCGATCTGGGCAGATCCGGGTAAA
TACGCCCTGATGGGAGCTGCTGCTCAGCTTGGTGGGATCGTGAGGATGAC
CCTTAGCCTGACAGTCATCATGATGGAGGCCACCAGCAACGTGACCTACG
GTTTTCCCATCATGTTGGTGCTGATGACTGCCAAGATTGTGGGTGATGTC
TTCATTGAGGGCCTCTATGACATGCACATCCAGCTGCAAAGTGTGCCCTT
CCTACACTGGGAAGCCCCGGTCACCTCACATTCGCTCACTGCCAGGGAAG
TAATGAGCACGCCTGTGACCTGCCTGAGGAGGAGAGAGAAGGTTGGCATC
ATCGTGGATGTCCTAAGTGACACAGCGTCTAATCACAATGGGTTCCCTGT
GGTGGAGGATGTAGGAGACACCCAGCCAGCCAGACTCCAAGGCCTAATCC
TGCGTTCCCAGCTCATCGTGCTCCTGAAGCACAAGGTGTTTGTGGAGAGG
TCCAACATGGGTTTGGTGCAGCGGAGACTGAGGCTGAAAGACTTTCGCGA
TGCCTACCCACGCTTCCCCCCAATCCAGTCCATCCACGTATCCCAGGATG
AGCGGGAGTGCACCATGGACCTTTCTGAGTTCATGAACCCTTCTCCCTAC
ACTGTGCCACAGGAGGCATCTCTTCCTCGAGTGTTCAAGCTGTTCCGGGC
TCTGGGCCTGAGGCACCTGGTCGTAGTAGACAACCACAATCAGGTGGTCG
GGCTGGTGACCAGGAAGGACCTAGCAAGATACCGCCTAGGAAAAGGAGGC
CTAGAAGAGCTTTCACTGGCCCAGACGTGAGGGCTGGCCCCCACCCTTGG
GCAGCGGCACCCCGGCCCCTCTGCACCTCCTCCCAGGGTCCCTGGTCTCA
GCCAAAGCCTTGCCCTGGGCAGTGCAGCAACAGGAGCAAATGCCCTCCCC
GGGCTTGGCTGGTGTGGGGCCCAGACCCTTTGTCCTGGGCAGTTGGTTTA
CATCATCAGCATTTCCCTATTCCCTGAACCTGCAGTCCTCAGACTTGTCC
CACTCCTGGGTCCCTTCTCCCAGGATGTAAAGTGTGTTTTCACACCCCTT

TABLE 4

Amino Acid Sequence of Rat CLC-7:
GenBank Accession No. NM113756

(SEQ ID NO:4)

MANVSKKVSWSGRDRDDEEGAPLLRRTGQPDEETPLLNGAGPGARQSHSA
LFRIGQMNNVELDDELLDPEVDPPHTFPKEIPHNEKLLSLKYESLDYDNS
ENQLFLEEERRINHTAFRTVEIKRWVICALLGWTGLVACFIIDIVVENLA
GLKYRVIKDNLDKFTEKGGLSFSLLLWATLNSAFVLVGSVIVAFTEPVAA
GSGIPQIKCFLNGVKIPHVVRLKTLVIKVSGVILSVVGGLAVGKEGPMII
ISGSVIAAGISQGRSTSLKRDFKIFEYFRRDTEKRDFVSAGAAAGVSAAF
GAPVGGVLFSLEEGASFWNQFLTWRIFFASMISTFTLNFVLSIYHGNMWD
LSSPGLINFGRFDSEKMAYTIHEIPVFLAMGVVGGILGAVFNALNYWLTM
FRIRYIHRPCLQVIEAMLVAAVTATVAFVLIYSSRDCQPLQGSSMSYPLQ
LFCADGEYNSMAAAFFNTPEKSVVSLFHDPPGSYNPMTLGLFTLVYFFLA
CWTYGLTVSAGVFIPSLLIGAAWGRLFGISMSYLTGAAIWADPGKYALMG
AAAQLGGIVRMTLSLTVIMMEATSNVTYGFPIMLVLMTAKIVGDVFLEGL
YDMHIQLQSVPFLHWEAPVTSHSLTAREVMSTPVTCLRRREKVGIIVDVL
SDTASNHNGFPVVEDVGDTQPARLQGLILRSQLIVLLKHKVFVERSNMGL
VQRRLRLKDFRDAYPRFPPIQSIHVSQDERECTMDLSEFMNPSPYTVPQE
ASLPRVFKLFRALGLRHLVVVDNHNQVVGLVTRKDLARYRLGKGGLEELS
LAQT

TABLE 5

Nucleotide Sequence of Mouse CLC-7:
GenBank Accession No. AK030444

(SEQ ID NO:5)

GAGATCACGTGAGACCTGGGCGCGGGTCACGGGAATGCTGCCGGTCTGCC
GGCTGTTCTTGTTGAGTTTGGTCCTCAGTGGGCCATGGCCAACGTCTCTA
AGAAAGTGTCTTGGTCCGGCCGAGATCGCGATGATGAGGAGGGGGCGCCA
CTGCTTCGAAGGACAGGGCAGCCTGACGAGGAGACGCCGCTGCTGAACGG
AGCTGGGCCGGGCGCGCGCCAGTCTCATTCTGCACTGTTCCGAATCGGAC
AGATGAACAACGTGGAGCTGGATGACGAGCTCCTGGACCCGGAAGTCGAT
CCTCCTCACACCTTCCCCAAGGAGATTCCACACAACGAGAAGCTCCTCTC
CCTCAAGTATGAGAGCCTGGACTATGACAACAGCGAGAATCAGCTCTTCC
TGGAGGAGGAAAGACGAATCAACCACACGGCTTTCCGGACAGTGGAGATC
AAGCGCTGGGTTATCTGTGCCCTCATTGGAATCCTCACAGGCCTAGTAGC
CTGCTTCATTGACATTGTAGTGGAGAACCTGGCAGGCCTCAAGTACCGCG
TCATCAAGGACAACATTGACAAGTTCACAGAGAAGGGCGGCCTGTCCTTC
TCCCTCCTGCTGTGGGCCACGCTGAACTCTGCCTTCGTACTCGTGGGGTC
TGTGATCGTGGCCTTCATAGAGCCTGTTGCTGCTGGCAGCGGAATCCCTC
AGATCAAGTGCTTCCTCAATGGGGTGAAGATCCCCCACGTGGTGCGGCTC
AAGACGCTGGTGATCAAGGTGTCCGGCGTGATTCTGTCTGTGGTAGGGGG
ACTGGCTGTGGGAAAGGAAGGGCCAATGATCCACTCAGGATCCGTGATAG
CTGCAGGGATTTCACAGGGAAGGTCAACATCACTCAAGCGAGATTTCAAG
ATCTTTGAATATTTCCGCAGAGATACAGAGAAGCGGGATTTTGTCTCAGC
TGGAGCTGCAGCTGGTGTATCTGCTGCATTTGGAGCCCCTGTGGGTGGGG
TCCTGTTTAGCTTGGAAGAGGGCGCCTCCTTCTGGAATCAGTTCCTGACT
TGGAGAATTTTCTTTGCTTCCATGATTTCCACCTTTACATTGAATTTTGT
TCTGAGCATCTACCATGGAAATATGTGGGACCTGTCCAGCCCTGGCCTCA
TAAATTTTGGAAGATTTGACTCAGAGAAAATGGCCTACACCATCCATGAG
ATTCCAGTCTTCATCGCCATGGGTGTGGTGGGTGGCATTCTTGGAGCTGT
GTTCAATGCCTTGAATTACTGGCTAACCATGTTTCGAATCAGGTACATCC
ACCGGCCCTGCCTCCAAGTGATTGAGGCCATGCTGGTGGCTGCTGTCACA
GCAACAGTTGCGTTTGTCTTGATTTACTCGTCTCGAGATTGCCAACCCCT
GCAGGGGAGCTCCATGTCCTACCCACTCCAGCTCTTCTGTGCAGATGGCG
AATACAACTCCATGGCCGCAGCCTTCTTTAACACCCCTGAGAAGAGCGTT
GTCAGCCTGTTCCATGACCCACCAGGCTCCTATAATCCCATGACTCTCGG
CCTGTTTACCCTGGTCTACTTCTTCCTGGCCTGCTGGACCTATGGCCTCA
CAGTATCTGCTGGTGTCTTCATCCCATCCCTGCTCATTGGGGCTGCCTGG
GGCCGACTCTTTGGCATCTCCCTGTCCTACCTCACAGGGGCAGCGATCTG
GGCAGATCCGGGTAAATACGCCCTGATGGGAGCTGCTGCTCAGCTTGGTG
GGATCGTGAGAATGACCCTTAGCCTGACAGTCATCATGATGGAGGCCACC
AGCAACGTGACCTACGGTTTCCCCATCATGTTGGTGCTGATGACTGCCAA
GATTGTGGGTGATGTTTTCATTGAGGGCCTCTATGACATGCACATCCAGC
TGCAGAGTGTACCCTTCCTACACTGGGAAGCCCCGGTCACCTCACACTCG
CTCACGGCCAGGGAAGTAATGAGCACACCTGTGACCTGCCTGAGGAGGCG
AGAGAAGGTTGGCATCATCGTGGATGTCCTAAGCGACACAGCGTCTAATC
ACAACGGATTCCCTGTGGTGGAGGATGTAGGAGACACCCAGCCAGCCAGG
CTCCAAGGCTTGATCCTGCGTTCCCAGCTTATCGTTCTCTTGAAGCATAA
GGTGTTTGTGGAGAGGTCCAACATGGGTTTGGTGCAGCGGCGACTGAGGC
TGAAGGACTTCCGAGATGCCTATCCACGCTTCCCCCCAATCCAGTCCATT
CATGTATCCCAGGATGAGCGTGAATGCACCATGGACCTTTCTGAATTCAT
GAACCCTTCTCCCTACACTGTGCCTCAGGAGGCATCTCTTCCTCGAGTGT
TCAAGCTCTTCCGGGCTCTGGGCCTGAGGCACCTGGTAGTAGTAGACAAC
CACAATCAGGTGGTCGGGCTGGTGACCAGGAAGGACCTAGCAAGATACCG
CCTAGGAAAGGGAGGCCTAGAAGAGCTTTCGCTGGCCCAGACGTGAGGGC
TGGCCCCCCAGCCTTGGACAGCAGTACCCCAGCCCCTCTGCACCTCCTCC
CAGGGTCCCTGATCTCAGCCAAAGCCTTGCCCCCTAGGCAGTGCAGCAAC
AGGAGCAAATGCCCTCCCTAGGCTTGGCTGGTGTGGGGGCCAGACCCTTT
GTCCTGGGCAGTTGGTTTCAGTCATCAGCACTTTCCCTATTCCCTGACCC
TCCAGGCCTCAGACTTGCCCCACTCCTGGTTCCTTCTCCCAGGATGAAGT
GTGTGTTATCACACCCCTTGTGGCTCACTTCTGAGAGCCAATAGAGTGTG
TGTGAGAAAGACAGACGTGAGCTACTACTTACTGCTGTCTCTGCTCAGAC
TCTGAGCCCTTCTCACCCTGTGTCTGCATTGCTCACACAGCCAGGGGCTT
CAGTAGCAGGGCATGGGGAGCTATACAGCCTGGAGCAGGCTCTGTCAGGG
AACCCACATGTGCCACACATGCCGCCCTGCATCTTTGTCCAGAAGATGGG
GAGACTGGTTCTGTACCTGGGGCTTGTGATCTGAAAATCACATGAAGACT
GAAATTGAATTCACCAACATCTGCCCTAGGCTTTCAGACCTGGGGTCATG
GCCCTGGACCACAGCCAGAAGGCAGGTCTTTTTGAGGCCCTTAGGTCCCT
GGGGTCCATAGTAGCTTGCTTACTACTGTACTGTGTGGTATGCCCTTGGG
AAGTGGCCTCCCAGGGCCTGGCCATCCTGGGGTAGCCCTAGGATGGATAG
ACCCGTGAGCACCAGAGCCAAGGAACTTCACCCCTGAACCCTGTGTTGTC
AAAGCTGGCCCCACTGACATCCTTCACTCACCTTCAGGGTCTTCTCCCCT
GGGAACTTTGTAAAGATAGTTGCAGTGTCTCTGGCACAAGTCTGTTATAA
GGTCACACAGGGTCTTTCTTTGGAAAGCTGGCAGCTCCTGCCTTTCAGTT
GTCCCTCCAAAAGAGCGGCTCTTCCTTCCATGAAGAGATGGGCTAAGTAT
GTTGAATCTGGAATGAGAAATGAGAGCCCCCTGTTCTCTGCCTTGGGCTC
AGCATTCTGGGGAGTCAGGCACTCAGCATGGTGATAAACAAGTGCAGATG
GCACTTTAAACAGTAGTTGCTGTCTGTGTCGGGCAGTGGTGCAGTTCTTG
AAGAGTACTGCAGCCAGCCCAGGGCCCAGTGGGAGGGCCGTCCAGGGACT
CTGGCCTTCTCGTCCCATCTCAGACTTGCTCTCAGACTCACTGCAGAGCA
TGGGTTCTGTGACCTGCTTGGATGGCATCCTGTCCTCAAGCCTGGCCACA
GCAGGCTCTTCAAGAAGTTAAAACTGACCCATCTGGTATAGAGGTCCAAG
TGACAACGCCAGGGGTGCTCTGGACCGGGAAGGGCCATTCCTAAAGTTGG
ACCCAAGGGGTGAAAAACACTGCACAGGGTCTCAAAGACTTGGTACCAAA
AAAGGCACGTGCAATCTGTCCACGGCTATGCAGTCTGTCCACAGCTGTGT
GCTGACTACATGCTGAAATGATATTTTTGGATATATTAGGTTAAATAAAA
CAATCAAAATTTGT

TABLE 6

Amino Acid Sequence of Mouse CLC-7:
GenBank Accession No. BAC26967

(SEQ ID NO:6)

MANVSKKVSWSGRDRDDEEGAPLLRRTGQPDEETPLLNGAGPGARQSHSA
LFRIGQMNNVELDDELLDPEVDPPHTFPKEIPHNEKLLSLKYESLDYDNS
ENQLFLEEERRINHTAFRTVEIKRWVICALIGILTGLVACFIDIVVENLA
GLKYRVIKDNIDKFTEKGGLSFSLLLWATLNSAFVLVGSVLVAFIEPVAA
GSGIPQIKCFLNGVKIPHVVRLKTLVIKVSGVILSVVGGLAVGKEGPMIH
SGSVIAAGISQGRSTSLKRDFKWEYFRRDTEKRDFVSAGAAAGVSAAFGA
PVGGVLFSLEEGASFWNQFLTWRWFASMISTFTLNFVLSIYHGNMWDLSS
PGLINFGRFDSEKMAYTIHEIPVFIAMGVVGGILGAVFNALNYWLTMFRI
RYIHRPCLQVIIEAMLVAAVTATVAFVLIYSSRDCQPLQGSSMSYPLQLF
CADGEYNSMAAAFFNTPEKSVVSLFHDPPGSYNPMTLGLFTLVYFFLACW
TYGLTVSAGVFIPSLLIGAAWGRLFGISLSYLTGAAIWADPGKYALMGAA
AQLGGIVRMTLSLTVIMMEATSNVTYGFPIMLVLMTAKIVGDVFIEGLYD
MHIQLQSVPFLHWEAPVTSHSLTAREVMSTPVTCLRRREKVGIIVDVLSD
TASNHNGFPVVEDVGDTQPARLQGLILRSQLIVLLKHKVFVERSNMGLVQ
RRLRLKDFRDAYPRFPPIQSIHVSQDERECTMDLSEFMNPSPYTVPQEAS
LPRVFKLFRALGLRHLVVVDNHNQVVGLVTRKDLARYRLGKGGLEELSLA
QT

Further, derivatives and homologues of CLC-7 may be used in the present invention. For example, nucleic acid sequences encoding CLC-7 of the present invention may be altered by substitutions, additions, or deletions that provide for functionally equivalent-conservative variants of CLC-7. For example, one or more amino acid residues within the sequence can be substituted by another amino acid of similar properties, such as, for example, positively charged amino acids (arginine, lysine, and histidine); negatively charged amino acids (aspartate and glutamate); polar neutral amino acids; and non-polar amino acids.

Other conservative amino acid substitutions can be taken from the Table 7, below.

TABLE 7
Conservative amino acid replacements

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D- Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| Phenylalanine | F | D-Phe, Tyr, D-Thr, L-Dopa, His, D-His, Trp, |
| | | D-Trp, Trans-D-Val 3,4, or 5-phenylproline, |
| | | cis-3,4, or 5-phenylproline |
| Proline | P | D-Pro, L-1-thioazolidine-4-carboxylic acid, D- |
| | | or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other analogs within the invention are those with modifications which increase protein stability; such analogs may contain, for example, one or more non-peptide bonds (which replace the peptide bonds) in the protein sequence. Also included are analogs that include residues other than naturally occurring L-amino acids, e.g., D-amino acids or non-naturally occurring or synthetic amino acids, e.g., β or γ amino acids.

CLC-7 as used in the present invention may be modified by, for example, phosphorylation, sulfation, acylation, or other protein modifications. It may also be modified with a label capable of providing a detectable signal, either directly or indirectly, including, but not limited to, radioisotopes and fluorescent compounds.

It will be apparent to one of skill in the art that screening assays, such as those set forth hereinbelow, may be used in methods of the present invention for the identification of CLC-7 modulators. Such modulators are useful in the treatment of osteoporosis.

Further, modulators found to affect CLC-7 activity may further be introduced into a murine or rat osteoporosis model, such as one which has been ovariectomized (which results in a situation similar to postmenopausal osteoporosis), in order to study the activity of such modulators in vivo. By way of example only, other murine model systems useful in the present invention for studying bone mass include those described in Matsushita, M., et al. (1986) Am. J. Pathol., 125:276-283 and Kuro-o M., et. al. (1997) Nature, 390:45-51.

An example of a known chloride inhibitor is niflumic acid, which is a non-specific chloride channel inhibitor which inhibits the acidification of mature osteoclasts.

In the present invention, techniques for screening large gene libraries may include cloning the gene library into replicable expression vectors, transforming appropriate cells with the resulting library of vectors, and expressing the genes under conditions for detection of a desired activity, e.g., binding of a protein to CLC-7 in the present invention. Techniques known in the art are amenable to high throughput analysis for screening large numbers of sequences created, e.g., by random mutagenesis techniques. Secondary screens can follow high throughput assays in order to identify further biological activities that will modulate the activity of CLC-7. The type of a secondary screen used will depend on the desired activity to be tested.

Compound screening assays are also provided in the present invention. In one aspect, the assay evaluates the ability of a compound to modulate activity of CLC-7. The term “modulating” encompasses enhancement, diminishment, activation or inactivation of CLC-7 activity. Assays useful to identify modulators of CLC-7 of the present invention, including peptides, proteins, small molecules, and antibodies that are capable of binding to CLC-7 and modulating its activity, are encompassed herein. A variety of assay formats may be used in the present invention and are known by those skilled in the art. Assays useful in the present invention are set forth hereinbelow.

In many drug-screening programs that test libraries of compounds and natural extracts, high throughput assays are desirable in order to maximize the number of compounds surveyed in a given period of time.

Compounds identified using assays, as discussed hereinabove, may be inhibitors or activators of CLC-7 and may bind to CLC-7, thereby modulating CLC-7 activity.

“CLC-7-associated disorders” refers to any disorder or disease state in which the CLC-7 protein plays a regulatory role in the physiological pathway of that disorder or disease. Such disorders or diseases include, but are not limited to, osteoporosis. As used herein the term “treating” refers to the alleviation of symptoms of a particular disorder in a patient, the improvement of an ascertainable measurement associated with a particular disorder, or the prevention of a particular immune, inflammatory or cellular response.

A compound which acts as a CLC-7 modulator may be administered for therapeutic use as a raw chemical or may be the active ingredient in a pharmaceutical formulation. Such formulations of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Compounds of the present invention may be administered by any suitable means, for example, orally, such as in the form of tablets, capsules, granules or powders; sublingually; buccally; parenterally, such as by subcutaneous, intravenous, intramuscular, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents.

Such compounds may, for example, be administered in a form suitable for immediate release or extended release. Immediate release or extended release may be achieved by the use of suitable pharmaceutical compositions comprising compounds of the present invention, or, particularly in the case of extended release, by the use of devices such as subcutaneous implants or osmotic pumps. Compounds of the present invention may also be administered liposomally.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art.

Compounds of the present invention may also be delivered through the oral cavity by sublingual and/or buccal administration. Molded tablets, compressed tablets or freeze-dried tablets are exemplary forms which may be used. Exemplary compositions include those formulating the compound(s) of the present invention with fast dissolving diluents such as mannitol, lactose, sucrose and/or cyclodextrins.

Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquefy and/or dissolve in the rectal cavity to release the drug.

Exemplary compositions for topical administration include a topical carrier such as Plastibase (mineral oil gelled with polyethylene).

The effective amount of a compound of the present invention may be determined by one of ordinary skill in the art, and includes exemplary dosage amounts for an adult human of from about 0.1 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to bone disorders.

The compounds of the present invention may be employed alone or in combination with each other and/or other suitable therapeutic agents useful in the treatment of bone disorders.

In another aspect, the present invention relates to the use of an isolated nucleic acid in “antisense or RNAi” therapy. As used herein, “antisense or RNAi” therapy refers to administration or in situ generation of oligonucleotides or their derivatives which specifically hybridize under cellular conditions with the cellular mRNA and/or genomic DNA encoding CLC-7 of the present invention so as to inhibit expression of the encoded protein, e.g., by inhibiting transcription and/or translation. In general, “antisense or RNAi” therapy refers to the range of techniques generally employed in the art, and includes any therapy that relies on specific binding to oligonucleotide sequences.

Gene constructs useful in antisense or RNAi therapy may be administered in any biologically effective carrier, e.g., any formulation or composition capable of effectively delivering a nucleic acid sequence to cells in vivo. Approaches include insertion of the subject gene in viral vectors including recombinant retroviruses, adenoviruses, adeno-associated viruses, and herpes simplex virus-1, or recombinant bacterial or eukaryotic plasmids. Viral vectors transfect cells directly; an advantage of infection of cells with a viral vector is that a large proportion of the targeted cells can receive the nucleic acid. Several viral delivery systems are known in the art and can be utilized by one practicing the present invention.

In addition to viral transfer methods, non-viral methods may also be employed. Most non-viral methods of gene transfer rely on normal mechanisms used by mammalian cells for the uptake and intracellular transport of macromolecules. Exemplary gene delivery systems of this type include liposomal derived systems, poly-lysine conjugates, and artificial viral envelopes. Nucleic acid sequences may also be introduced to cell(s) by direct injection of the gene construct or by electroporation.

In clinical settings, the gene delivery systems can be introduced into a patient by any of a number of methods, each of which is known in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells which produce the gene delivery system.

The following section sets forth materials and methods used in the present invention, and which were utilized in the Examples set forth hereinbelow.

### Materials and Methods

1. Cell Culture and RNA Isolation

Raw264.7 cells (American Type Culture Collection; Rockville, Md.) were cultured in high glucose Dulbecco's modified eagle's medium (DMEM; GIBCO/BRL) containing 10% FCS. Recombinant soluble RANKL (Gst-RANKL) was obtained by fusing the extracellular domain of human RANKL (amino acids 158-317) to glutathione-S-transferase (Gst) and expression in E. coli. Total RNA was extracted from Raw264.7 cells treated with media alone or with media supplemented with 100 ng/ml Gst-RANKL for various time up to five days. Total RNA was isolated with the RNeasy Midi/Maxi kit (Qiagen) according to the manufacturer.

2. Chip Hybridization and Data Analysis

The cRNA preparation and array hybridization was performed according to the Affymetrix protocol (Affymetrix, CA). cRNA was prepared from 10 ug of total RNA. The RNA was denatured at 70° C. with T7-tagged oligo-dT primers and then reverse transcribed with Superscript II (GIBCO BRL) at 42° C. for 1 hr. Second-strand cDNA was synthesized by adding DNA pol I, E. coli DNA ligase and RNase H, and incubation was carried out for 2 hrs at 16° C. After being extracted once with phenol/chloroform, the synthesized cDNA was used for in vitro transcription with a BioArray High Yield RNA Transcript Labeling Kit (Enzo). Labeled cRNA was purified with RNeasy columns (Qiagen) and then fragmented (10 ug/per chip) before hybridization.

Two types of Affymetrix murine chips were used in these studies. Mu6500 set contains approximately 6,500 genes and EST sequences, while Mu11K set contains approximately 11,000 genes and ESTs. The oligo array cartridges were prehybridized at 45° C. for 10 min. The cRNA samples were added to cartridges and the hybridization was performed for 16 hrs at 45° C. with 60 rpm rotation. After hybridization, the chips were washed and stained in a fluidics station using the antibody amplification protocol from Affymetrix. The chips were then scanned using a Hewlett-Packard GeneArray scanner. The data was analyzed using GeneChip software (Affymetrix). An intensity value and presence/absence (P/A) call was derived from hybridization signal for each gene to represent its expression level. For cluster analysis, Self-Organizing Map software from MIT was used.

3. Real-time Quantitative RT-PCR

To verify the genes identified to be differentially expressed by the Affymetrix chip experiment, an independent method, real time semi-quantitative RT-PCR, was used. Biosystems 5700 sequence detection system was used for this purpose. The system measures fluorescence in real time during the PCR reactions. As the PCR products increase in quantity throughout the PCR cycling, the fluorescence intensity increases proportionally. PCR primers were designed to analyze each sequence of interest. Primer sets were as follows: for CLC-7 chloride channel, the primers are PY1618 (5′-tgctctcagactcactgcaga-3′) (SEQ ID NO:7), and PY1619 (5′-gttgtcacttggacctctat-3′) (SEQ ID NO:8).

The experiment measures the relative abundance of the transcript of interest among all the samples (control as well as RANKL-treated samples). The data are normalized by comparison to the expression of two non-differentially expressed genes (house-keeping genes): mouse ribosomal protein S9 (primers: PY314 (5′-gagggcaagatgaagctgga-3′) (SEQ ID NO:9) and PY315 (5′-ggatgttcaccacctgcttg-3′) (SEQ ID NO:10)) and mouse glyceraldehyde-3-phosphate dehydrogenase (primers: PY283 (5′-cactggtgctgccaaggctg-3′) (SEQ ID NO:11) and PY284 (5′-cttgctcagtgtccttgctgg-3′) (SEQ ID NO:12).

4. Osteoclast Differentiation Assay

Raw264.7 cells were plated at 1×10⁴ cells/48-well and cultured in presence or absence of 100 ng/ml Gst-RANKL for four to five days. At the end of the culture period, the cells were examined for typical markers of the osteoclast lineage including multinucleation, formation of resorption lacunae on dentine slices, positive staining for tartrate resistant acid phosphatase (TRAP) and expression of genes such as TRAP, cathepsin K and c-src.

As illustrated in the following Examples, it is now found in the present invention that CLC-7 is associated with osteoclast differentiation and activation and, accordingly, clinical osteoporosis.

### EXAMPLE 1

### Verification of the Raw264 Cell Model and Affymetrix Chip Technology

In order to verify the utility of this in vitro model and to verify the Affymetrix chip technology, a preliminary transcription profiling experiment was performed using RNA samples isolated from RANKL-treated Raw264 cells. Raw264 cells were treated with either vehicle or RANKL for 2 and 5 days. Poly A⁺ RNAs were isolated at the end of the treatment. Probes were generated from these RNAs and used to hybridize Affymetrix chip Mu6500, which contained approximately 6400 murine genes and EST sequences. After washing, the arrays were scanned and values were assigned to each gene representing its expression level. The expressional change was measured by comparing the intensity of each gene with or without RANKL treatment. Many genes were identified as upregulated by RANKL treatment.

To verify the result from Affymetrix chip experiments, more than 20 genes with desired expression profiles were chosen for real-time quantitative PCR analysis. As shown in Table 8, the PCR and Affymetrix results show similar expression patterns. Table 9 sets for the descriptions of the genes shown in Table 8. Of the genes verified by PCR, TRAP, gelatinase B, carbonic anhydrase, and cathepsin K exhibit increases in expression after incubation of Raw264 cells with RANKL for 2 and 5 days. These genes have been demonstrated to be involved in osteoclast differentiation and resorption.

TABLE 8
PCR and Affymetrix Expression Results

| | Ratio of RANKL-treated vs control | | | | |
|---|---|---|---|---|---|
| | Affy, | | Affy, | | |
| Gene | 2 d | PCR, 2 d | 5 d | PCR, 5 d | Summary |
| M99054 | 92 | 13 | 165 | 47 | increase, at 2, 5 day |
| D12712 | 1 | 40 | 116 | 67 | increase, at 2, 5 day |
| W82261 | 20 | 6 | 2 | 2 | increase, at 2, 5 day |
| K00811 | 12 | 10 | 6 | 2 | increase, at 2, 5 day |
| W13263 | 5 | 4 | 10 | 7 | increase, at 2, 5 day |
| X66119 | 14 | 23 | 2 | 2 | increase, at 2, 5 day |
| L35302 | 5 | 4 | 3 | 1 | increase, at 2 day |
| AA109799 | 0.5 | 1 | 8 | 5 | increase, at 5 day |
| M12660 | 0.09 | 0.09 | 0.10 | 0.12 | decrease, at 2, 5 day |
| U18869 | 0.11 | 0.13 | 0.20 | 0.30 | decrease, at 2, 5 day |

TABLE 9
Description of Genes from PCR and Affymetrix Expression Analysis

| Gene | Description |
|---|---|
| M99054 | TRAP, (clone lambda-MG5.3) acid phosphatase type 5 |
| | gene, complete cds |
| D12712 | mRNA for type IV collagenase (gelatinase B) |
| W82261 | Homologous to sp Q02547: VACUOLAR ATP |
| | SYNTHASE SUBUNIT AC39 (32 KD ACCESSORY |
| | PROTEIN) |
| K00811 | carbonic anhydrase isozyme II mRNA, complete cds |
| W13263 | Homologous to sp P43236: CATHEPSIN K PRECURSOR |
| | (EC 3.4.22.−) (OC-2 PROTEIN). |
| X66119 | serotonin transporter (SET) gene, promoter region and |
| | partial cds |
| L35302 | TNF receptor associated factor 1 (TRAF1) mRNA, |
| | complete cds |
| AA109799 | Homologous to sp P30551: CHOLECYSTOKININ TYPE |
| | A RECEPTOR (CCK-A RECEPTOR). |
| M12660 | CFh locus, complement protein H gene, complete cds, |
| | clones MH(4,8) |
| U18869 | mitogen-responsive 96 kDa phosphoprotein p96 mRNA, |
| | alternatively spliced p67 mRNA |

These results demonstrate that the data from the in vitro Raw264 model is in agreement with the published literature based on various osteoclast models. The PCR analysis also demonstrates that the Affymetrix chip technology is reliable to be used as a platform for large-scale gene expression analysis.

### EXAMPLE 2

### CLC-7 Identification Using Affymetrix Mu11K Chip

Probes were generated from RNAs isolated from Raw264 cells treated with RANKL for various periods of time (control: 6h, 1d, and 3d; RANKL-treatment: 3h, 6h, 16h, 1d, 2d, 3d, and 4d) and used to hybridize Affymetrix Mu11K chips, which contain approximately 11,000 genes with known functions as well as EST sequences.

After washing, the arrays were scanned and values were assigned to each gene representing its expression level. Genes and EST sequences that showed a relative change of more than 3 fold were identified. EST sequences without any annotation were subjected to an extensive BLAST analysis, which resulted in the identification of sequence homology to known proteins. Subsequent analysis of genes with annotation of channel related function resulted in the discovery that CLC-7 was upregulated during osteoclast differentiation, as shown in Table 10.

TABLE 10
Upregulation of CLC-7 During Osteoclast Differentiation.

| | | peak time of | fold of |
|---|---|---|---|
| Gene | Description | induction | change |
| W99907 | CLC-7 chloride channel | 3, 4 d | 4.3 |

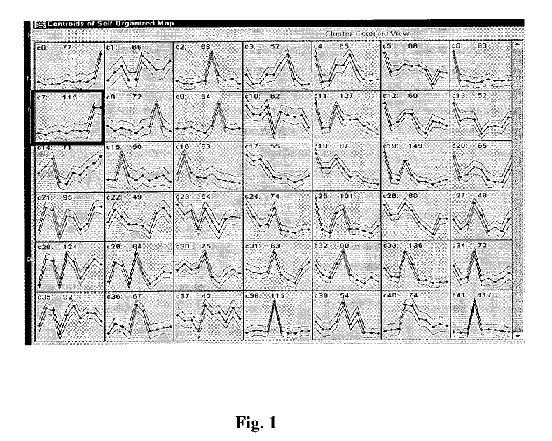

Cluster analysis of expression data allows us to group genes into different clusters according to the similarity of their expression pattern. Genes in the same cluster tend to have similar function, or are in the same complex, or are involved in the same process or pathway. Using Self-Organization Map analysis tool, genes from osteoclast differentiation and activation process were grouped into 42 clusters, as shown in FIG. 1. Significantly, CLC-7 are grouped in the same cluster (115 genes in Cluster 7, indicated by black box) as cathepsin K and vacuolar ATPase subunits, which are important for the resorption of the bone. Thus, this result demonstrates that CLC-7 channel is coupled with vacuolar ATPase in the acidification process.

Based on the up-regulation and cluster analysis, CLC-7 is shown to be important in bone resorption. It is known that Cl⁻ currents mediate charge compensation during the electrogenic H+ transport into the resorption microenvironment by vacuolar ATPase. Thus the activation of the chloride current is very important for the acidification and bone resorption. As shown in Table 10, CLC-7 is upregulated at later stages of differentiation (3 to 5 days after RANKL induction). At these stages, the multinuclear osteoclasts are able to resorb bone in in vitro models. These results indicated that CLC-7 is one of the chloride channels responsible for the Cl⁻ currents in osteoclasts during the acidification and resorption process. Thus, inhibition of this channel will reduce the acidification and bone resorption in vitro as well as in vivo.

### EXAMPLE 3

### PCR Verification of CLC-7 Expression

The upregulation of CLC-7 in late differentiation was verified by real-time RT-PCR. The channel is upregulated by 3- to 5-fold, 3 to 5 days after RANKL induction, consistent with the results shown in Example 2.

### EXAMPLE 4

### Screening for Modulators of the CLC-7 Chloride Channel

A high throughput (HTP) primary screen is useful for screening for inhibitors of CLC-7. Once compounds are identified, a lower throughput secondary assay is useful to confirm the results.

A. HTP Primary Screen

CLC-7 is expressed in the mouse mature osteoclasts derived from Raw264.7 cell line. Accordingly, Raw264.7-derived osteoclasts can be used to screen for compounds that inhibit CLC-7. Alternatively, native or mutated CLC-7 protein can be over-expressed by plasmid transfection in a heterologous cell line such as HEK-293, COS-7, or CHO-K1 cells or in a native cell line such as Raw264.7. Once a CLC-7-expressing cell line is generated, it is used to screen chemical libraries for compounds that inhibit activity of the chloride channel. Two different assays can be used to measure CLC-7 activity, as follows:

(i) Chloride Ion Transport

CLC-7-mediated chloride transport can be measured by using a halide-sensitive fluorescence probe, such as MQAE (American. J. Physiol. 275, C1048). As MQAE fluorescence is quenched by chloride ions in a dose-dependent fashion, compounds that interfere with channel activity can be identified by the change in the fluorescence intensity. This assay can be run in HTP mode on a multiwell fluorescence plate reader.

(ii) Membrane Potential Changes

Activation of chloride channels changes the membrane potential of cells in which they are expressed. Fluorescent indicators of cell membrane potential are known in the art and can be used to detect channel activity (e.g., DIBAC, (1994) Chem. Phys. Lipids 69:137-150). Compounds that interfere with the channel activity will impact the effects of the channel on membrane potential.

B. Low Throughput Secondary Screen

CLC-7 inhibitors identified in the cell-based primary screen are further tested in a secondary assay utilizing expressed channels and cellular electrophysiology. The chloride currents are measured using two-electrode voltage clamp (Xenopus oocytes) or patch clamp (mammalian cell lines) techniques.

C. In Vitro Bone Resorption Assay

The ability of these compounds to inhibit bone resorption by osteoclasts is measured by in vitro assays. Dentin slices may be used to quantify resorption pit area and volume. Osteologic plates are used to quantify resorption pit area generated by osteoclasts with or without compounds. Alternatively, the resorbed bone particles may be used to quantify peptide released by osteoclasts.

D. Identification of Subunits that Interact with the CLC-7 Channel

Coimmunoprecipitation of proteins in Raw264.7 cells or primary osteoclasts with antibodies against CLC-7 can be used to identify other proteins that interact with CLC-7 to form a functional chloride channel or to regulate its activity. Alternatively, yeast two-hybrid interaction cloning can be applied to identify proteins that interact with CLC-7 channel.

### EXAMPLE 5

### RNAi Interference Experiment

RNAi interference experimentation was conducted to confirm the role of the CLC-7 channel in bone resorption.

For RNAi transfection and bone resorption, the experiment was performed as follows: 1×10⁴ Raw264.7 cells were seeded per 48 well and were cultured for 4 days with 100 ng/ml RANKL. The RNAi reagent was obtained from Sequitur. On day 4, the cells were transfected with 150 nM RNAi and 2 μg/ml lipofectamine overnight. The next day, the medium was replaced and 10 mg of powered bovine cortical bone particles (PelFreeze, Inc) were distributed into each well (James, I. E., et al. (1999) J. Bone Miner. Res., 14:1562-9). The particles were resuspended in 200%1 of tissue culture media and incubated at 4° C. for 24 h storage. 200 μl bone particles were added to the precultured cells. The bone particles were allowed to settle for 10 min, and the cells were cultured for another 48 h at which time 100 μl of the supernatant was removed for the C-telopeptide Crosslaps™ ELISA analysis according to the manufacturer's instructions (Nordic Bioscience/Denmark). The absorbance was measured at 450 nm (Molecular Devices, Sunnyvale, Calif.).

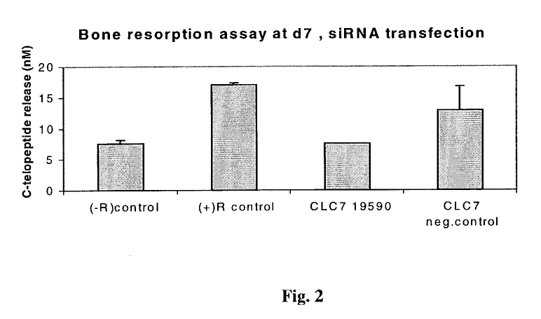

The data shows that the addition of RANKL results in the increase of bone resorption at day 7 (2.2 fold, FIG. 2). This is due to the induction of Raw264 cell differentiation and activation by RANKL. When the RNAi was added to the RANKL-induced cells at day 4, there is a slight decrease in bone resorption for the negative control RNAi. This is likely due to a non-specific inhibition. But when RNAi CLC-7 19590, a specific RNAi for CLC-7 sequence, was added, there is a 41% decrease in bone resorption (compared to the CLC-7 negative control). This result confirms the importance of CLC-7 in bone resorption.

### EXAMPLE 6

### Expression and Cellular Localization of CLC-7 and other CLC Chloride Channels

A. Cellular Localization of CLC-7 Chloride Channel in Various Cell Types by CLC-7-GFP Plasmid

1. CLC-7 in Raw264.7 Cells

RAW264.7 is a monocytic cell line that can be induced with RANKL to form mature osteoclast-like cells. CLC-7 is endogenously expressed in this cell line. A gfp tagged human CLC-7 was overexpressed in this cell line in order to examine the localization of the channel in the osteoclast precursor.

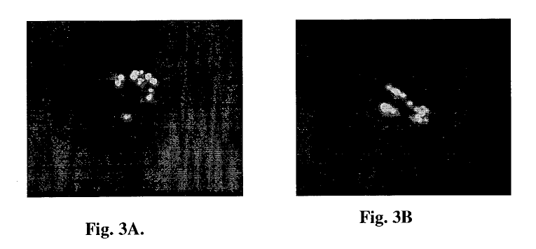

1 ug CLC-7-GFP in pcDNA 3.1 TOPO was transfected into 10⁶ undifferentiated RAW264.7 cells with the Amaxa electroporator using program U-14. 2.5×10⁵ cells were plated per well of a 24 well plate in 500 uL complete media. 24 hrs post-transfection, media was removed and replaced with 500 uL complete media containing 200 ng/ml RANKL. Images were captured on the Zeiss META 510 confocal microscope (488 Argon laser to excite GFP), and the results are shown in FIGS. 3A (10× objective; 48 hrs. post transfection) and 3B (10× objective; 72 hrs. post transfection).

2. CLC-7 in Retina Pigment Epithelia (RPE) Cells

CLC-7 knockout mice exhibit retinal degeneration. The localization of a gfp tagged and overexpressed CLC-7 was examined in RPE cells, a cell line commonly used in models of retinal degeneration.

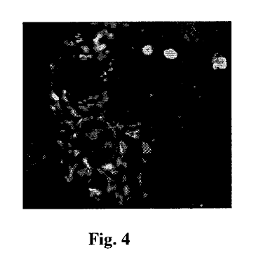

300 ng CLC-7-GFP in pcDNA 3.1 TOPO was transfected into RPE cells (1.5×10⁵ cells plated 24 hrs. per well of an 8 well polylysine coated chamber slide 24 hours before transfecting) with 3 ug/ml Lipofectamine 2000. 24 hrs post-transfection, the nuclei were stained with 5 uM DRAQ5. DRAQ5 was diluted in complete media and placed on cells for 15 minutes at 37° C. Following 2 washes with 1×PBS, cells were fixed for 30′ at 4° C. in 3% paraformaldehyde containing 0.1% Triton X-100. Following 2 washes with 1×PBS, slides were coverslipped. Images were captured on the Zeiss META 510 confocal microscope (488 Argon laser to excite GFP, 633 HeNe2 laser to excite DRAQ5) using a 40× water objective, as shown in FIG. 4.

3. CLC-7 in HEK293 Cells

Moderate levels of CLC-7 mRNA expression have been demonstrated in the human embryonic cell line HEK293. The localization of a gfp tagged and overexpressed CLC-7 was examined in HEK293.

1 ug CLC-7-GFP in pcDNA 3.1 TOPO was transfected into HEK293 cells (105 cells plated 24 hrs. per well of an 8 well polylysine coated chamber slide 24 hours before transfecting) with 3 ug/ml Lipofectamine 2000. 24 hrs post-transfection, the nuclei were stained with SuM DRAQ5. DRAQ5 was diluted in complete media and placed on cells for 15 minutes at 37° C. Following 2 washes with 1×PBS, cells were fixed for 30′ at 4° C. in 3% paraformaldehyde containing 0.1% Triton X-100.

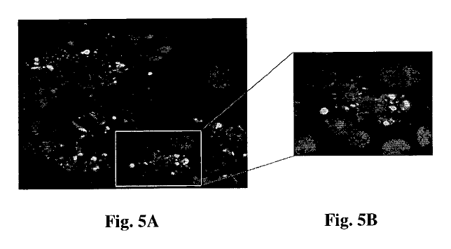

Following 2 washes with 1×PBS, slides were coverslipped. Images were captured on the Zeiss META 510 confocal microscope (488 Argon laser to excite GFP, 633 HeNe2 laser to excite DRAQ5), as shown in FIGS. 5A (40× water objective) and 5B (5× digital zoom of boxed region in FIG. 5A).

The staining pattern observed in all three cell lines for gfp-tagged CLC-7 indicates that CLC-7 is predominantly localized to intracellular vesicles. This distribution accounts for the lack of chloride conductance observed in the plasma membrane of CLC-7 transfected HEK293. However, in mature actively resorbing osteoclasts these vesicles may fuse with the plasma membrane, thereby conferring the chloride conductance that is required for maintaining electroneutrality during extracellular acidification.

B. Expression of CLC-7 and Other CLCs in Raw264 Cells by PCR.

RNA was isolated and purified from RAW264 cells grown in the presence of 100 ng/ml RANK-Ligand for 0, 2 or 5 days. Samples were prepared for Taqman analysis by digesting 2.5 ug of RNA with 3 ul DNase (Ambion) for 1 hour at 37° C., followed by cDNA synthesis with Superscript II reverse transcriptase (Invitrogen). cDNA samples were analyzed by Taqman analysis on an Applied Biosystems 7900 Sequence Detection System using the default PCR settings.

Primer sets used are listed in Table 11. Conditions for Taqman PCR were as follows: 300 nM primers, 200 nM probe, 4.5 mM MgCl₂, 1× Platimun Quantitative PCR Mix (Invitrogen) with ROX. All samples were analyzed in duplicate, and expression levels relative to ribosomal protein L-30 were determined by the 2^{-ΔΔxτ} method.

TABLE 11

Primer Sets

| Gene of | | | |
|---|---|---|---|
| Interest | Forward Primer | Reverse Primer | Probe |
| CLC-2 | Cgagatgagtcctgaggagatctt | cgattttgcagtcactgaagttg | tgggaagaacagcagctagatgagccat |
| | (SEQ ID NO:11) | (SEQ ID NO:12) | (SEQ ID NO:13) |
| CLC-3 | Gtcattattgttgcagccattactg | tcactggtgttgagccttgtg | tgtgatagccttccc* |
| | (SEQ ID NO:14) | (SEQ ID NO:15) | (SEQ ID NO:16) |
| CLC-4 | Tggttctcttttacgtggagtatcat | cataaacccccaaagactccaa | ctggtacatggctgaactcttccctttcat |
| | (SEQ ID NO:17) | (SEQ ID NO:18) | (SEQ ID NO:19) |
| CLC-5 | ggtgtcggaagcgtaaaacc | tggccgtcacaatgagtacct | cagttgggcaagtat* |
| | (SEQ ID NO:20) | (SEQ ID NO:21) | (SEQ ID NO:22) |
| CLC-6 | ctgggagccacattcaactgt | ttaggtttcgggtgcacgtt | acaagaggcttgcaaagtaccgtatgcg |
| | (SEQ ID NO:23) | (SEQ ID NO:24) | (SEQ ID NO:25) |
| CLC-7 | aggctccaaggcttgatcct | cacaaacaccttatgcttcaagaga | cgttcccagcttatc* |
| | (SEQ ID NO:26) | (SEQ ID NO:27) | (SEQ ID NO:28) |
| L-30 | ggaagtacgtgctgggctaca | ccaacttcgctttgccttgt | cagactctgaagatgatca* |
| | (SEQ ID NO:29) | (SEQ ID NO:30) | (SEQ ID NO:31) |

* Denotes Taqman MGB Probe

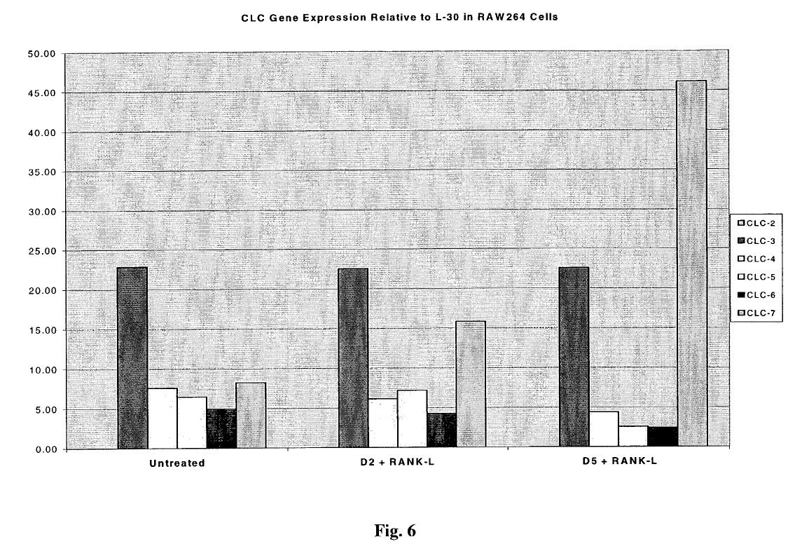

The results are shown in FIG. 6. As demonstrated in other cell types, CLC-3 is the predominantly expressed CLC in undifferentiated RAW264 cells. However, when stimulated to differentiate with RANKL, CLC-7 expression increases almost 2-fold by Day 2 and over 5-fold by Day 5. CLC-3 expression did not change at Day 5, while CLC-4, -5, and -6 each decreased approximately 50%. It is clear, therefore, that CLC-7 is the only CLC whose expression increases in RAW264 cells following RANKL stimulation.

C. Gene Expression of CLC-7 in Various Tissues by PCR

The mRNA expression of human CLC-7 in various human tissues, including heart, liver, kidney, brain, skeletal muscle, placenta, spleen and retina, were investigated using TaqMan PCR. Additionally, the gene expression in different cell lines, including HEK 293, CHO-K1, human osteoclast and RAW263 cells, were investigated. The results show that CLC-7 is ubiquitously expressed in all of above tissues and cell lines.

### EXAMPLE 7

### 10 Establishment of Stable CLC-7 HEK 293 Cell Lines

By using mammalian expression vector, pTV1.5 (BMS), human CLC-7 cDNA was transfected into HEK293 cells. Under selection with G418, ten stable cell lines were selected. Clones number 4, 5, and 10 expressed high levels of CLC-7 RNA compared to control HEK-293 cells.

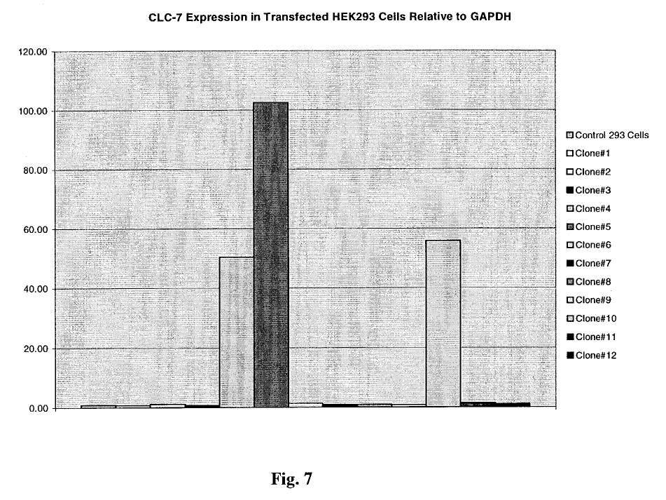

Clonal colonies of HEK-293 cells transfected with CLC-7 in a mammalian expression plasmid were analyzed by Taqman PCR to determine CLC-7 expression levels. Primers and conditions were as described for the RAW cell analysis, supra. CLC-7 expression was normalized to GAPDH. The results are shown in FIG. 7.

Clones 5 and 10 were further analyzed by Western blotting to examine protein expression. Equal amounts of total cell homogenate (10 ug) were loaded on a gel and blotted to nitrocellulose membrane. After blocking with 5% BSA/0.1% Tween 20, the blots were incubated with an antibody to the C-terminus of CLC-7 (Santa Cruz Biotechnology, 1:300 dilution), washed, and incubated with an alkaline phosphatase conjugated antibody to goat IgG (1:3000, BioRad). Washed blots were then developed with the ImmuneStar AP Substrate Pack (BioRad) and exposed to film.

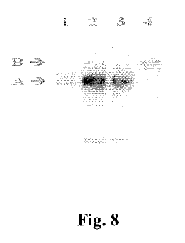

The result is shown in FIG. 8. Lane 1 contains control HEK-293 cell homogenate, Lane 2 Clone #5, Lane 3 Clone #10 and Lane 4 homogenate from cells transiently transfected with a CLC-7/GFP chimeric protein. Clearly Clones # 5 and 10 are overexpressing CLC-7 protein (Arrow A). Consistent with the RNA data, Clone #5 (Lane 2) appears to express a greater amount of CLC-7 than Clone #10 (Lane #3). Arrow B denotes the larger, CLC-7/GFP chimera in Lane 4. The results demonstrate the overexpression of CLC-7 RNA and protein in stably transfected HEK-293 cells. Therefore, we have established a high expression stable human CLC-7 HEK293 cell line. The cell line could be useful for CLC-7 assay development.

Clone #5 was further analyzed by immunolocalization to determine where the overexpressed CLC-7 resides in the cell. Cells were fixed for 15 min at 4° in 3% paraformaldehyde/0.1% Triton X-100, washed and blocked for 60 min at 37° C. in 3% BSA/5% Goat Serum/5% FBS in PBS. Primary antibodies to CLC-7 (Santa Cruz Biotechnology, 1:200) and LAMP-1 (Antibody H4A3, Developmental Studies Hybridoma Bank, ascites fluid at 1:1000) were diluted in blocking buffer and added to the cells for 1-1.5 hours at 37°. After washing with PBS, secondary antibodies diluted in blocking buffer were added for 1 hour at 37°. For CLC-7 localization, a FITC labeled anti-goat antibody was used at 1:400, for LAMP-1, a rhodamine conjugated anti-mouse antibody was used at 1:100. Samples were washed with PBS and visualized on a confocal microscope.

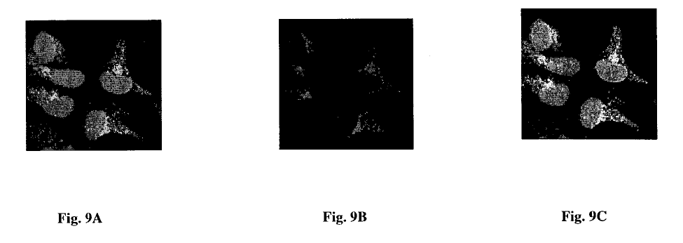

Cells in FIG. 9A were stained for LAMP-1. The antibody recognizes an antigen located in numerous large vesicles in the cytoplasm and perinuclear region. Since LAMP-1 is a lysosomal marker, the vesicles are therefore identified as such. Cells in FIG. 9B were stained for CLC-7. This antibody also stained numerous, large cytoplasmic vesicles. FIG. 9C represents an overlay of FIG. 9A and FIG. 9B. Yellow staining represents areas where CLC-7 and LAMP-1 co-localize. This data demonstrates that CLC-7 is localized in lysosomal vesicles in stable cell lines overexpressing CLC-7.

While the invention has been described in connection with specific embodiments therefore, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. All references cited herein are expressly incorporated in their entirety.

What is claimed is:

1. An assay for identifying a compound that modulates the activity of the chloride channel CLC-7, comprising:
(a) providing a cell expressing CLC-7;
(b) contacting said cell expressing CLC-7 with a test compound; and
(c) determining whether said test compound modulates the activity of CLC-7.

2. The assay of claim 1, wherein said assay is a cell-based assay.

3. The assay of claim 1, wherein said assay is a cell-free assay.

4. The assay of claim 3, wherein said cell-free assay is a detached patch clamping.

5. The assay of claim 1, wherein said test compound modulates the activity of CLC-7.

6. The assay of claim 1, wherein said test compound is a CLC-7 activator.

7. The assay of claim 1, wherein said test compound is a CLC-7 inhibitor.

8. The assay of claim 1, wherein said test compound binds to CLC-7.

9. The assay of claim 1, wherein said assay is for identifying compounds which are useful for the treatment of osteoporosis.

10. A method for the treatment of osteoporosis, comprising administering to a patient in need thereof a therapeutically effective amount of a compound identified by the assay of claim 1.

11. A method for the treatment of osteoporosis, comprising:
(a) identifying a patient having osteoporosis; and
(b) administering to said patient a therapeutically effective amount of a modulator of CLC-7.

12. The method of claim 11, wherein said modulator is a CLC-7 inhibitor.

13. The method of claim 11, wherein said modulator is a CLC-7 activator.

14. A method for the prevention of osteoporosis, comprising:
(a) identifying a patient at risk for osteoporosis; and
(b) administering to said patient a therapeutically effective amount of a modulator of CLC-7.

15. The method of claim 14, wherein said modulator is a CLC-7 activator.

16. The method of claim 14, wherein said modulator is a CLC-7 inhibitor.

17. A method of decreasing the differentiation of osteoclast precursor cells into osteoclast cells or decreasing the activity of mature osteoclasts, comprising contacting said osteoclast precursor cells with a CLC-7 modulator.

18. The method of claim 17, wherein said modulator is a CLC-7 inhibitor.

19. The method of claim 17, wherein said modulator is a CLC-7 activator.

20. A compound capable of modulating the activity of CLC-7.

21. The compound of claim 20, wherein said compound is identified by:
(a) providing a cell expressing CLC-7;
(b) contacting said cell expressing CLC-7 with said compound; and
(c) determining whether said compound modulates the activity of CLC-7.

22. The compound of claim 20, wherein said compound is a CLC-inhibitor.

23. The compound of claim 20, wherein said compound is a CLC-7 activator.

24. The compound of claim 20, wherein said compound binds to CLC-7.
